(19) United States
(12) Patent Application Publication
(10) Pub. No.: US 2026/0240537 A1
(43) Pub. Date: Aug. 20, 2026

(54) REUSABLE AUTOMATED SUTURE ADAPTER FOR LAPAROSCOPIC SURGERY

(71) Applicant: KOC UNIVERSITESI, Istanbul (TR)

(72) Inventors: Serdar AYDIN, Istanbul (TR); Mert YASLI, Istanbul (TR)

(73) Assignee: KOC UNIVERSITESI, Istanbul (TR)

(21) Appl. No.: 19/470,834

(22) PCT Filed: Mar. 11, 2024
(86) PCT No.: PCT/TR2024/050239

§ 371 Date: Sep. 30, 2025

(30) Foreign Application Priority Data
Apr. 4, 2023 (TR) 2023/003676

(51) Int. Cl. A61B 17/04

(52) U.S. Cl. CPC A61B 17/0469; A61B 17/0491

(57) ABSTRACT

A reusable automated suture adapter consisting of two different parts is designed for use in laparoscopic surgery. Furthermore, the adapter makes suturing easier, faster, and safer by being attached to the jaws of a laparoscopic bipolar grasper.

### CROSS REFERENCE TO THE RELATED APPLICATIONS

This application is the national phase entry of International Application No. PCT/TR2024/050239, filed on Mar. 11, 2024, which is based upon and claims priority to Turkish Patent Application No. 2023/003676, filed on Apr. 4, 2023, the entire contents of which are incorporated herein by reference.

### TECHNICAL FIELD

The present invention relates to a reusable and elastic automated suture adapter consisting of two different parts, which is designed for use in laparoscopic surgery. Furthermore, the present invention relates to the adapter making suturing easier, faster, and safer by being attached to the jaws of a laparoscopic bipolar grasper.

### BACKGROUND

Laparoscopic surgery is a minimally invasive surgical method for performing intra-body procedures by making small incisions and using a small camera and specialized instruments. Laparoscopic surgery can be used in many different types of surgery, such as gallbladder surgery, appendectomy, treatment of gynecological problems such as ovarian cysts or tumors, treatment of gastrointestinal problems such as gastro-esophageal reflux disease, stomach ulcers, intestinal obstruction or diverticulitis, and other conditions that require surgical intervention, such as biliary stricture. This method is less painful and has a faster recovery time compared to open surgery. It also has advantages such as less blood loss, less tissue damage, and less risk of infection.

Pelvic organ prolapse (POP) is a condition where the female genital organs descend below their normal position due to age, vaginal deliveries, patient's weight, hysterectomy, connective tissue diseases, and dysfunction of the levator ani muscle. In some patients, this condition does not cause any symptoms; however, in some patients, complications such as urinary and fecal incontinence and protrusion of the vagina towards the outer opening can be seen due to this condition. The anterior wall of the vagina is usually the structure which is most exposed to prolapse. In this case, the bladder descends lower than normal, and urinary incontinence complaint is more common in these patients. POP has a highly negative impact on the patient's health and quality of life [1].

Sacrospinous ligament fixation is a surgical technique used for the treatment of pelvic organ prolapse. In this surgical technique, the posterior vaginal wall is stitched to the sacrospinous ligament and its descent is prevented [2]. There are open and closed operation techniques. The open surgical technique is performed by dissecting the posterior vaginal wall and stitching it directly to the sacrospinous wall. The open surgical technique is less preferred than before due to urinary dysuria and the risk of recurrence of POP [2]. The closed surgical technique is performed by exposing the labia minora located at the entrance of the vagina through minimal incision and stitching the posterior vaginal wall to the sacrospinous ligament without being dissected, with laparoscopic surgery using the blind dissection method. The disadvantage of this technique is that the surgical site cannot be adequately visualized and therefore the possibility of injury increases [3].

When the technique was first introduced, sacrospinous ligament fixation surgery was performed by dissecting the posterior vaginal wall using a long dissecting scissors through the vaginal opening and stitching the posterior wall to the sacrospinous ligament transvaginally. The currently adapted technique has adopted the method of making a small incision in the labia minora at the vaginal opening and entering the pelvic cavity through the blind dissection of this incision and stitching the posterior vaginal wall to the sacrospinous ligament. Although this method is more risk-free than the first method, blind dissection and then stitching pose a risk for a surgeon without manual skills. The suture needle should be passed through both the posterior vaginal wall and the sacrospinous ligament; however, it can be challenging to perform this process at a site with limited field of view. A mistake, which can be made at the site, can complicate the healing process, impact the success of the surgery, and also cause different complications. It is therefore important to reduce the risk of this process.

Today, the cost of designs, which can be used as automated suture adapters, poses a problem for making these devices widespread. Since the cost for developing and manufacturing a device specific to the case and the technique will be high, surgeons prefer blind dissection and increase the risk. In addition to the high cost of the device, since it is disposable, it further increases the cost.

In the known state of the art, there are similar applications which have been developed against the above-mentioned problems. For example, the instrument called Autosuture Endo Stitch® allows the needle to be transferred between the two heads of a trocar handle by the needle mechanism located on the end of the trocar. In this way, when the end of the trocar is closed on the tissue through which the needle is intended to pass, the needle is passed through the desired tissue, thus facilitating suturing. The advantage of this method is that it enables a safer transfer of the needle and makes the surgery easier. By means of this instrument, the possibility of injury to the hypogastric venous plexus and hemorrhoid plexus veins, which has been reported as a complication, has been reduced [4]. Its disadvantage is that this device has a structure too long for the operation and its length should be decreased [5].

Another device which has been developed is a suture instrument called Capio® Slim. The working principle of this instrument is based on the fact that a hook with a needle located on its end grasps the sacrospinous ligament and the needle is passed through the ligament with one hand. It has been found that this method reduces the possibility of bleeding, decreases postoperative pain, and increases the success of surgery by nearly 30% compared to Miya hook, which is an older technique [6].

Another device is a suture instrument called EnPlace®. It can be said that the working principle of this instrument, unlike other instruments, is to puncture the posterior wall intravaginally and then the sacrospinous ligament, thus passing the thread through both structures [7].

U.S. Pat. No. 8,257,366 B2, an application known in the state of the art, discloses a digital suture fixation system used in endoscopic surgery and a method performed by using this system. The system disclosed in the patent document is realized by being attached to the finger of a specialist who will apply the technique.

U.S. Pat. No. 10,299,828 B2, an application known in the state of the art, discloses a system for transvaginal sacrospinous ligament fixation, comprising a one-piece suture portion configured to affix a vaginal wall to a sacrospinous ligament.

U.S. Pat. No. 8,282,657 B2, an application known in the state of the art, discloses a suturing assembly configured to place suture in tissue, comprising a handle having an actuator, a shaft connected to the handle, and a one-piece head coupled to the shaft.

### SUMMARY

The objective of the invention relates to the automated suture adapter consisting of two different parts according to the invention making suturing easier, faster, and safer by being attached to the two ends of a laparoscopic grasping instrument, which is called trocar used in laparoscopic surgery and has the function of grasping. The parts of the adapter according to the present invention are preferably detachable.

A further objective of the invention is to reduce complications associated with sutures after surgery by standardizing the suturing process.

Another objective of the invention is to increase the success of the surgery by reducing the possibility of injury during surgical operation by means of the automated suture adapter designed for use in laparoscopic surgery.

The invention relates to performing sacrospinous ligament fixation surgery by using the automated suture adapter of the present invention preferably together with other instruments and a camera (by applying separately or simultaneously) in a laparoscopic surgical procedure. The present invention is for use in sacrospinous ligament fixation surgeries.

The suture adapter disclosed in the present invention allows surgeons to easily suture the areas in the abdomen that are hard to reach.

### BRIEF DESCRIPTION OF THE DRAWINGS

“A Reusable Automated Suture Adapter for Laparoscopic Surgery”, which is developed to fulfill the objective of the present invention, is illustrated in the accompanying figures, in which:

FIGS. 1A-1C are the illustrations of the female part from different angles (FIG. 1A: Perspective view, FIG. 1B: Perspective view, and FIG. 1C: Open view).

FIGS. 2A-2E are the illustrations of the male part from different angles (FIG. 2A: Perspective view, FIG. 2B: Perspective view, FIG. 2C: Side view, FIG. 2D: Front view, and FIG. 2E: Top and bottom view).

FIG. 3 is the illustration of the laparoscopic grasper (A).

FIG. 4 is the close-up illustration of the jaws (A¹, A¹¹) of the laparoscopic bipolar grasper.

### DETAILED DESCRIPTION OF THE EMBODIMENTS

For the convenience of the reader, the numbers of the parts, which are shown in the drawings with brief description above and referred to in the specification, are given below.
A. Laparoscopic grasper
   A¹. Lower jaw (for the female part)
   A¹¹. Upper jaw (for the male part)
B. Suture Thread
1. Female part:
   11: upper surface
   12: lower surface
   13: side surface
   14: side surface
   15: front surface
   16: back surface
2. Male part
   21: upper surface
   22: lower surface
   23: side surface
   24: side surface
   25: front surface
   26: back surface
3. Nut insertion area (cavity) for the screw
   31: Nut insertion area (cavity) for the screw in the female part
   32: Nut insertion area (cavity) for the screw in the male part
4. Phallic portion (in the male part)
   41: notch (for grasping and pulling the suture thread)
5. Passage (in the female part)
6. Cavity in the female part into which the phallic portion enters
7. Cavity (passage) in the part for attaching the jaw to the part
   71: Cavity (passage) in the female part for attaching the lower jaw to the female part
   72: Cavity (passage) in the male part for attaching the lower jaw to the male part

The present invention relates to an automated suture adapter used in laparoscopic surgical applications.

In the present invention, the term “stitch” or “suture” means a process carried out for ensuring the integrity of tissues, which have been damaged in any way, and accelerating their recovery. This process is intended for hemostasis (ligation of major vascular structures and tissue pedicles) or mutual approximation of the disrupted tissue during the surgical procedure.

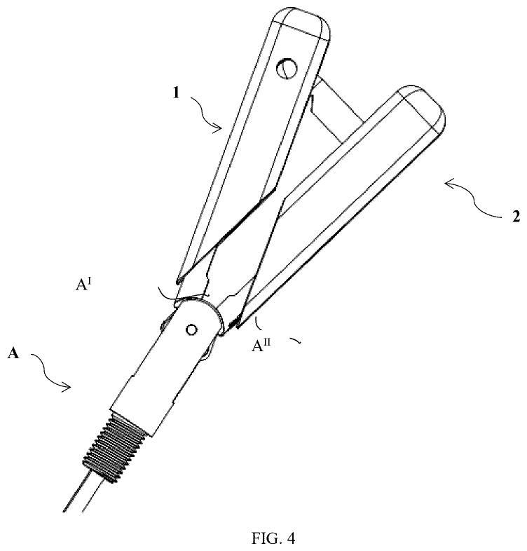

In present invention, the term “laparoscopic grasper” is known in the art as laparoscopic needle grasper. These needle graspers are used to grasp and manipulate suture needles to enable suturing without contact with hands during laparoscopic processes. As can be seen in patent no US20140358163A1, there are grasper parts which remain outside the body and jaw parts which perform the manipulation inside the body. In the U.S. Pat. No. 10,039,591 B2, the corrugated structure of the jaw part can be seen. The illustrations in FIGS. 3 and 4 show the laparoscopic grasper and jaws before the suture adapter of the present invention is attached to the grasper.

The knowledge and skill/experience of a specialist is generally important in the laparoscopic suturing and knotting techniques known today. It is a time-consuming process that requires advanced skill. By means of the automated suture adapter of the present invention, there is no dependence on the skill of a specialist with this regard, and it is also enabled that the process can be performed quickly and easily. Also, one of the technical advantages of the suture adapter of the present invention is that it is automated compared to techniques that are used manually by being controlled with hands.

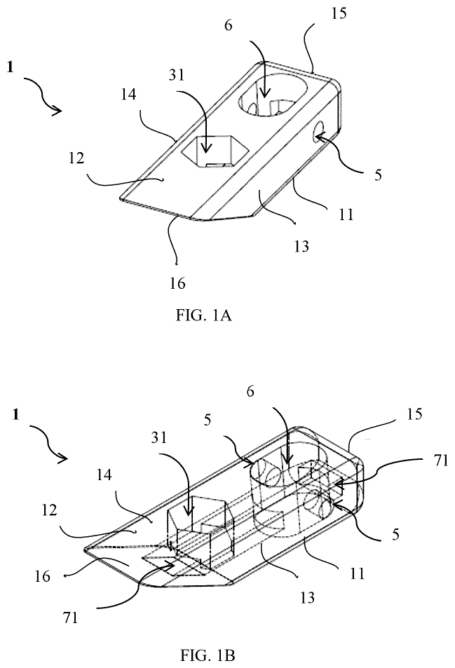

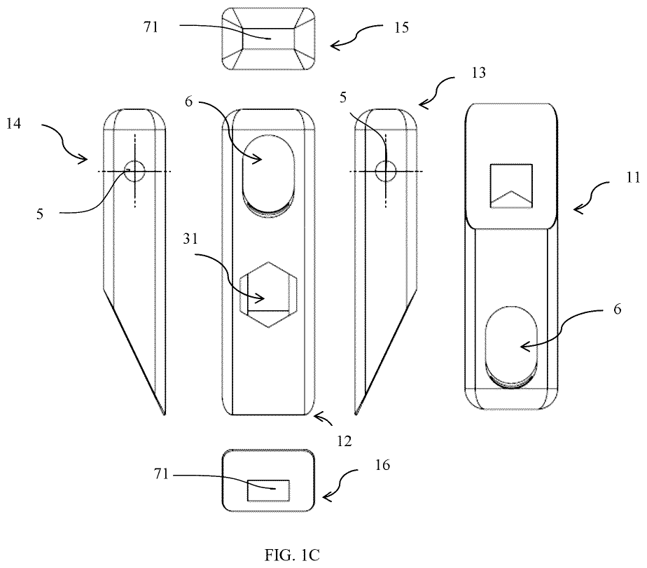

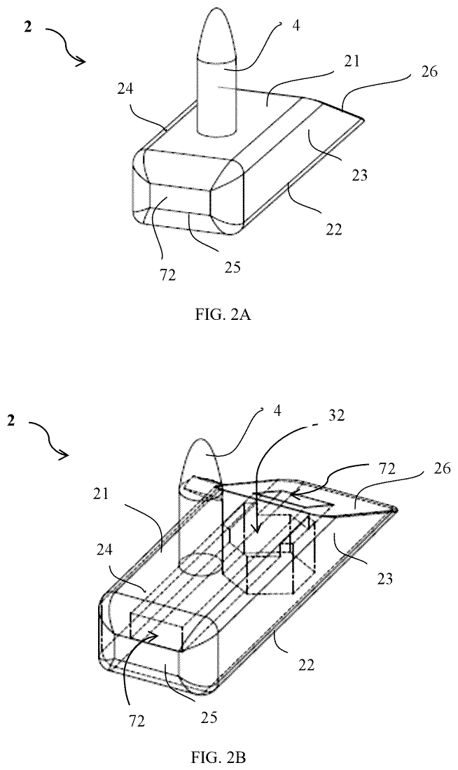

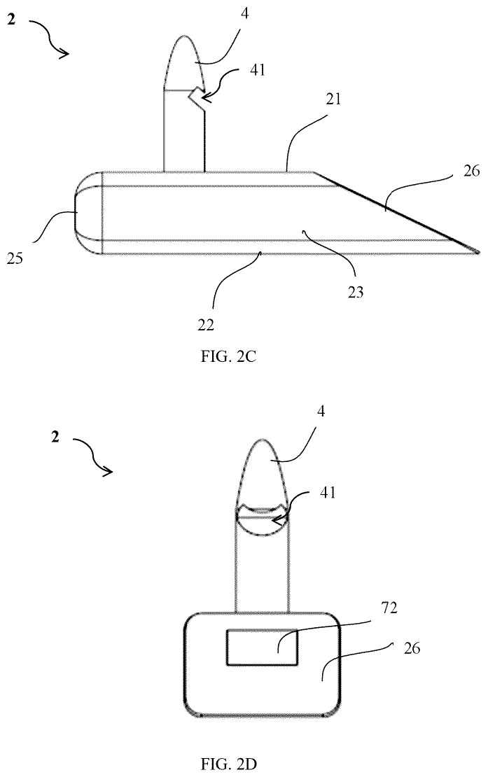

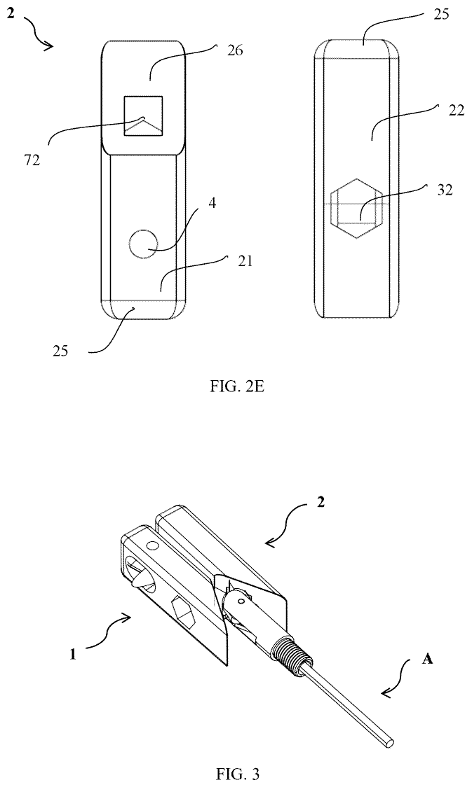

The present invention relates to a laparoscopic suture adapter (suture device) with two parts, namely a female part (FIGS. 1A-1C) and a male part (FIGS. 2A-2E), and a method of using the said suture adapter. The design of the parts may vary according to specific needs; however, they have the same basic structure as described herein. Preferably, the female (1) and male (2) parts are not connected to each other at one end but are two separate parts.

In the said invention, both parts (1 (female part), 2 (male part)) comprise at least one insertion area (31 (for the female part), 32 (for the male part)) for fixing the parts from their lower surfaces (12, 22) to the corresponding surfaces. The said corresponding surfaces (A¹, A¹¹), to which the female (1) and male (2) parts are fixed, are preferably connected or in contact with each other at one end, and they are surfaces at least one of which is movable, such that they move towards or against each other, like scissors; or one of them is fixed and the other is movable. This movement enables the suturing process to be realized.

In the preferred embodiment of the invention, the corresponding surfaces (A¹, A¹¹) are the lower (A¹) and upper (A¹¹) jaws of a laparoscopic bipolar grasper (A). Preferably, it is a bipolar grasper (A). Preferably, the upper jaw (A¹¹) is movable relative to the lower jaw (A¹) (the lower jaw (A¹) can be fixed) or both jaws (A¹, A¹¹) are movable. In the preferred embodiment of the invention, the female part (1) and the male part (2) of the suture adapter are attached and inserted into the lower jaw (A¹) and the upper jaw (A¹¹), respectively, and then fixed thereto.

In the preferred embodiment of the invention, for fixing the female (1) and male (2) parts to the corresponding jaws (A¹, A¹¹), the insertion areas (31, 32) in the lower surfaces of the parts (1, 2) are such that the insertion areas (31, 32) are of a size that can accommodate a nut. Preferably, the nut used herein is the M3 nut having dimensions and shape known in the art. The nut inlet of the adapter is designed in M3 size into which a nut can fit universally, thus ensuring ease of use.

As in FIGS. 2A-2E, the male part (2) has an upper surface (21) comprising a phallic portion (4), which is rectangular and resembles a suture needle, and which has the shape of a cylindrical body having a rounded or sharp end with an upwardly angled and narrowing cut for piercing the tissue. The phallic portion (4) is a fixed protrusion on the upper surface (21), preferably in the shape of a suture needle. In FIGS. 2A-2E, there are shown the lower surface (22), the side surfaces (23, 24), the front surface (25) and the inclined back surface (26) of the male part (2), which are in contact when it is fixed to the upper jaw (A¹¹).

As in FIGS. 1A-1C, the female part has two circular holes parallel to each other on both side surfaces (13, 14) and passage (5) located between the two parallel holes, through which the suture thread passes. There are also two interconnected holes in the shape of straight slits which are located at a slight angle on the upper (11) and lower (12) surfaces and allow the passage of the phallic portion (4) of the male part (2). In other words, said two interconnected holes essentially form a cavity (6) of a size into which the phallic portion (4) of the male part (2) can enter. Since this cavity (6) has an angle of 10 degrees, it facilitates the entry of the phallic portion (4). In FIGS. 1A-1C, there are shown the lower surface (12), the side surfaces (13, 14), the front surface (15) and the inclined back surface (16) of the female part (1), which are in contact when it is fixed to the lower jaw (A¹).

As a working principle, these two parts (1, 2) of the suture adapter are fixed by being screwed from the jaw portions (A¹, A¹¹) of the laparoscopic grasper (A) such that their upper surfaces (11, 21) face each other. In other words, they are fixed by being screwed such that the lower surfaces of both parts (12, 22) are in contact with the jaw portions (A¹, A¹¹).

When the jaws (A¹, A¹¹) of the laparoscopic grasper (A) are closed by the manipulation of the surgeon (i.e., moved closer to each other), the two parts (1, 2) contact each other and the phallic portion (4) passes through the cavity (passage) (6) in the female part (1) and collides with the suture thread (B) that is passing through the two circular holes parallel to each other and the passage (5) located between the holes. When the jaws (A¹, A¹¹) of the laparoscopic grasper (A) are opened (i.e., moved away from each other), the male part (2) separates from the female part (1) and pulls the suture thread (B) towards itself by means of a notch (41) located on the phallic portion during the process.

In a preferred embodiment of the invention, by attaching the automated suture adapter to the jaws (A¹, A¹¹) of the laparoscopic bipolar grasper (A), it is ensured that the laparoscopic grasper (A) is used as the handle of the suture adapter.

The suture adapter disclosed in the present invention is manufactured from a biocompatible and elastic material suitable for laparoscopic surgical applications in order to make this adapter reusable. The adapter is made of any biocompatible material which can be easily cleaned and sterilized by known methods according to the technical field.

Three-dimensional (3D) printing is a developing field due to the use of various novel materials and printing techniques (8). There are many fields where this technology can be used. One of these fields is the production of case-specific medical devices which are durable, high-quality, and cost-effective. In a preferred embodiment of the invention, an automated and reusable suture adapter is obtained by being designed for 3D printing and manufactured with 3D printing application. In 3D printing application, the material used for manufacturing the invention is metal. Here, the invention is realized with a low-cost and ergonomic design by means of 3D printing.

In a preferred embodiment of the invention, the adapter can be produced by metal printing, if desired and required, and thus it becomes reusable. Preferably, the material used in the invention, which is determined to be suitable for contact with body fluids or tissues, is metal. In the said suture adapter, the jaws of the laparoscopic bipolar grasper (A) are in the shape of a hollow frame as shown in FIG. 4. To be able to fix the female (1) and male (2) parts to the jaws (A¹, A¹¹) by means of the screw, the jaws (A¹, A¹¹) are preferably first passed through the cavities (71, 72), which are located in the parts (1, 2) and correspond to the shape of the jaws. These cavities are the cavities (71 (for the female part), 72 (for the male part)) which are formed such that the holes located in the front and back surfaces (15, 16) of the female part (1) are interconnected and the holes located in the front and back surfaces (25, 26) of the male part (2) are interconnected.

The fact that the jaws (A¹, A¹¹) are in the shape of a frame and hollow enables the suture adapter to perform its task easily when the jaws (A¹, A¹¹) are opened and closed. The back surfaces (16, 26) of the parts into which the jaws (A¹, A¹¹) enter, are designed to be inclined and thus, when the jaws (A¹, A¹¹) are closed, the female and male parts (1, 2) overlap and the male part (2) is superimposed on the female part (1) on the plane of the female part (1). Thus, the phallic portion (4) of the male part (2) pulls the thread (B) that is passing through the female part (1) to its side and automates the suturing process. The male part (4) performs this process of pulling and grasping the thread by means of a notch (41), i.e., a recess, on the phallic portion (4).

In the present invention, by smoothing the edges of the parts (1, 2), they are prevented from traumatizing the tissue. By means of the adapter of the present invention which is designed specifically for this surgical procedure, both the risk of complication and the cost have been reduced.

The said invention can be manufactured by using standard techniques or production methods known in the state of the art.

With regard to these basic concepts, it is possible to develop a wide variety of applications by means of “A Reusable Automated Suture Adapter for Laparoscopic Surgery” of the present invention and the invention, which is essentially as defined in the claims, cannot be limited to examples disclosed herein.

### REFERENCES

[1] Jelovsek JE, Maher C, Barber MD. Pelvic organ prolapse. Lancet. 2007; 369(9566):1027-38.
[2] Favre-Inhofer A, Carbonnel M, Murtada R, Revaux A, Asmar J, Ayoubi JM. Sacrospinous ligament fixation: medium and long-term anatomical results, functional and quality of life results. BMC Womens Health. 2021; 21(1):66.
[3] Leron E, Erez O, Shwarzmam P, Baessler K. Sacrospinous ligament fixation (SSLF): an old method with new horizons. Arch Gynecol Obstet. 2022; 305(6):1379-82.
[4] Morley GW, DeLancey JO. Sacrospinous ligament fixation for eversion of the vagina. Am J Obstet Gynecol. 1988; 158(4):872-81.
[5] Schlesinger RE. Vaginal sacrospinous ligament fixation with the Autosuture Endostitch device. Am J Obstet Gynecol. 1997; 176(6):1358-62.
[6] Mowat A, Wong V, Goh J, Krause H, Pelecanos A, Higgs P. A descriptive study on the efficacy and complications of the Capio (Boston Scientific) suturing device for sacrospinous ligament fixation. Aust N Z J Obstet Gynaecol. 2018; 58(1):119-24.
[7] Ben Zvi M, Weintraub AY, Friedman T, Neuman M, Tsivian A. Minimally invasive meshless and minimal dissection ligament fixation system for apical organ prolapse procedures: A 4-year prospective follow up study. Int J Gynaecol Obstet. 2022; 158(3):657-62.
[8] Salmi M. Additive Manufacturing Processes in Medical Applications. Materials (Basel). 2021; 14(1). Epub 20210103. doi: 10.3390/ma14010191. PubMed PMID: 33401601; PubMed Central PMCID: PMC7796413.

What is claimed is:

1. An automated suture adapter, fixed to a laparoscopic bipolar grasper having a lower jaw and an upper jaw, for use in laparoscopic surgery, comprising a female part and a male part, wherein:
the female part comprises:
an upper surface, a lower surface, side surfaces, a front surface and an inclined back surface,
a first insertion area for fixing the female part from the lower surface of the female part to the lower jaw,
a first cavity of a size, wherein a phallic portion in the male part is configured to enter into the first cavity, and
two circular holes parallel to each other on both side surfaces and a passage located between the two circular holes, wherein a suture thread passes through the two circular holes;
the male part comprises:
an upper surface, a lower surface, side surfaces, a front surface and an inclined back surface,
a second insertion area for fixing the male part from its the lower surface of the male part to the lower jaw, and
the phallic portion in a shape of a cylindrical body, wherein a first end of the phallic portion is in contact with the upper surface of the male part, and a second end of the phallic portion is rounded or sharp with an angled and narrowing cut for piercing, and a notch on the phallic portion.

2. The automated suture adapter according to claim 1, wherein a process of fixing the automated suture adapter to the laparoscopic bipolar grasper is performed by attaching and inserting the female part into the lower jaw and the male part into the upper jaw.

3. The automated suture adapter according to claim 1, wherein before a process of fixing the automated suture adapter to the laparoscopic bipolar grasper, the lower jaw and the upper jaw are passed through second cavities, wherein the second cavities are located in the female part and the male part and correspond to shapes of the lower jaw and the upper jaw.

4. The automated suture adapter according to claim 1, wherein a process of fixing the automated suture adapter to the laparoscopic bipolar grasper is performed by fitting a nut into the first insertion areas in the female part and the second insertion area in the male part and fixing to the lower jaw and the upper jaw by a screw.

5. The automated suture adapter according to claim 1, wherein a size of the first cavity located in the female part is of a allowed to accommodate the phallic portion, and the first cavity has an angle of 10 degrees for facilitating entry of the phallic portion.

6. The automated suture adapter according to claim 1, wherein the automated suture adapter has a working principle as follows:
when the lower jaw and the upper jaw of the laparoscopic bipolar grasper are closed:
the phallic portion of the male part passes into the first cavity in the female part and passes through the two circular holes and collides with the suture thread, and
the phallic portion of the male part passes through the passage located in the female part and collides with the suture thread;
when the lower jaw and the upper jaw of the laparoscopic bipolar grasper are opened:
the male part separates from the female part and pulls the suture thread towards by the notch located on the phallic portion during a process.

7. The automated suture adapter according to claim 1, wherein the automated suture adapter is made of any material determined to be suitable for contact with body fluids or tissues.

8. The automated suture adapter according to claim 7, wherein the material is metal.

9. The automated suture adapter according to claim 2, wherein a process of fixing the automated suture adapter to the laparoscopic bipolar grasper is performed by fitting a nut into the first insertion area in the female part and the second insertion area in the male part and fixing to the lower jaw and the upper jaw by a screw.

10. The automated suture adapter according to claim 3, wherein a process of fixing the automated suture adapter to the laparoscopic bipolar grasper is performed by fitting a nut into the first insertion area in the female part and the second insertion area in the male part and fixing to the lower jaw and the upper jaw by a screw.

11. The automated suture adapter according to claim 2, wherein a size of the first cavity located in the female part is allowed to accommodate the phallic portion, and the first cavity has an angle of 10 degrees for facilitating entry of the phallic portion.

12. The automated suture adapter according to claim 3, wherein a size of the first cavity located in the female part is allowed to accommodate the phallic portion, and the first cavity has an angle of 10 degrees for facilitating entry of the phallic portion.

13. The automated suture adapter according to claim 4, wherein a size of the first cavity located in the female part is allowed to accommodate the phallic portion, and the first cavity has an angle of 10 degrees for facilitating entry of the phallic portion.

14. The automated suture adapter according to claim 2, wherein the automated suture adapter has a working principle as follows:
when the lower jaw and the upper jaw of the laparoscopic bipolar grasper are closed:
the phallic portion of the male part passes into the first cavity in the female part and passes through the two circular holes and collides with the suture thread, and
the phallic portion of the male part passes through the passage located in the female part and collides with the suture thread;
when the lower jaw and the upper jaw of the laparoscopic bipolar grasper are opened:
the male part separates from the female part and pulls the suture thread towards by the notch located on the phallic portion during a process.

15. The automated suture adapter according to claim 3, wherein the automated suture adapter has a working principle as follows:
when the lower jaw and the upper jaw of the laparoscopic bipolar grasper are closed:
the phallic portion of the male part passes into the first cavity in the female part and passes through the two circular holes and collides with the suture thread, and
the phallic portion of the male part passes through the passage located in the female part and collides with the suture thread;
when the lower jaw and the upper jaw of the laparoscopic bipolar grasper are opened:
the male part separates from the female part and pulls the suture thread towards by the notch located on the phallic portion during a process.

16. The automated suture adapter according to claim 4, wherein the automated suture adapter has a working principle as follows:
when the lower jaw and the upper jaw of the laparoscopic bipolar grasper are closed:
the phallic portion of the male part passes into the first cavity in the female part and passes through the two circular holes and collides with the suture thread, and
the phallic portion of the male part passes through the passage located in the female part and collides with the suture thread;
when the lower jaw and the upper jaw of the laparoscopic bipolar grasper are opened:
the male part separates from the female part and pulls the suture thread towards by the notch located on the phallic portion during a process.

17. The automated suture adapter according to claim 5, wherein the automated suture adapter has a working principle as follows:
when the lower jaw and the upper jaw of the laparoscopic bipolar grasper are closed:
the phallic portion of the male part passes into the first cavity in the female part and passes through the two circular holes and collides with the suture thread, and
the phallic portion of the male part passes through the passage located in the female part and collides with the suture thread;
when the lower jaw and the upper jaw of the laparoscopic bipolar grasper are opened:
the male part separates from the female part and pulls the suture thread towards by the notch located on the phallic portion during a process.

18. The automated suture adapter according to claim 2, wherein the automated suture adapter is made of any material determined to be suitable for contact with body fluids or tissues.

19. The automated suture adapter according to claim 3, wherein the automated suture adapter is made of any material determined to be suitable for contact with body fluids or tissues.

20. The automated suture adapter according to claim 4, wherein the automated suture adapter is made of any material determined to be suitable for contact with body fluids or tissues.
